(19) United States
(12) Patent Application Publication
(10) Pub. No.: US 2008/0254490 A1
(43) Pub. Date: Oct. 16, 2008

(54) Salivary Protease Assays for Identifying Increased Risk of Preterm Delivery Induced by Premature Rupture of Fetal Membranes

(76) Inventor: Ramkumar Menon, Nashville, TN (US)

Correspondence Address: Law Office of Kenneth K. Sharples, Sena Plaza Building - Suite 54, 125 East Palace Avenue, Santa Fe, NM, 87501, US

(21) Appl. No.: 11/883,669

(22) PCT Filed: Feb. 2, 2006
(86) PCT No.: PCT/US2006/003807

§ 371 Date: Jun. 10, 2008

Related U.S. Application Data
(60) Provisional application No. 60/649,519, filed on Feb. 2, 2005.

(51) Int. Cl. G01N 33/573; C12Q 1/37; G01N 33/53

(52) U.S. Cl. 435/7.92; 435/23; 435/7.1

(57) ABSTRACT

Assay methods for identifying an increased risk of preterm delivery induced by premature rupture of the fetal membranes are provided. More specifically, the invention provides salivary protease assays indicative of amniochorion and fetal membrane concentrations of proteases involved in the degradation of the fetal membranes leading to rupture. An increase in salivary protease levels beyond normal or individual baseline provides an indication that protease levels in fetal membranes and/or the amniotic fluid are abnormally elevated. Abnormally elevated protease levels define an increased risk of a premature delivery caused by rupture of the fetal membranes. Thus, abnormally high levels of salivary protease levels indicates elevated risk of PROM, and provides an indication for therapeutic intervention to prevent PROM-induced preterm labor.

### BACKGROUND OF THE INVENTION

In the United States, one in ten pregnancies result in premature birth with all the consequent health and educational implications associated with this complication. Prematurity is the third leading cause of perinatal death. 500,000 babies are born prematurely each year in the United States and more than 100,000 neonatal fatalities are reported due to prematurity. More than 70% of the survivors are expected to have neurological and physical handicaps. Preterm premature rupture of the membranes (PROM) is directly antecedent to 30% to 50% of all preterm births and occurs in approximately 6% to 8% of women before 37 weeks of completed gestation. The incidence of PROM and subsequent premature delivery remains high due, at least in part, to the lack of a specific diagnostic test to predict PROM. A predictive biological marker for PROM has yet to be identified and a lack of diagnostic test(s) to predict this condition enhances the seriousness of this event that generally results in premature birth with consequent high rate of neonatal morbidity and mortality.

A number of major etiologic factors have been linked to the onset of PROM. Weakening of the amniochorion extracellular matrix (ECM) by collagen degradation is one of the key events predisposing to rupture. Endogenous and exogenous factors activate collagen degradation. The endogenous factors include a local variation in membrane thickness and a reduction in collagen content. The exogenous factors include effects of bacterial metabolism or a host or fetal inflammatory response. Although bacterial collagenases are detected in the amniotic fluid (AF) during PROM, they are neither specific nor produced in sufficient quantities to degrade human collagens. Infection induced activation of membranes matrix specific enzymes (matrix metalloproteinases-MMPs) have recently been shown to be associated with excessive collagen turnover and membrane weakening leading to rupture. Increased concentrations of active forms of MMPs have been documented in the AF and in the membranes after PROM.

Traditional tests (e.g., fern tests, funneling, litmus tests, etc.) are used to confirm and diagnose PROM after it has occurred; however, no tests exist to predict PROM before it happens. Therefore, there is a great need for a simple and effective assay predictive of PROM and/or a high risk of PROM.

### SUMMARY OF THE INVENTION

The invention relates to assay methods for identifying an increased risk of preterm delivery induced by premature rupture of the fetal membranes. More specifically, the invention provides salivary protease assays indicative of amniochorion and fetal membrane concentrations of proteases involved in the degradation of the fetal membranes leading to rupture. In one embodiment, total salivary protease activity is measured using a gelatin substrate. In other embodiments, particular proteases implicated in PROM (i.e., matrix metalloproteases, including MMP-9) are measured in saliva, using assay formats described herein and/or those widely known in the art. An increase in protease levels beyond normal or individual baseline, particularly in matrix metalloproteases, in the saliva of a pregnant patient (see Examples 2 and 3, infra), provides an indication that protease levels in fetal membranes and/or the amniotic fluid are abnormally elevated. Abnormally elevated protease levels define an increased risk of a premature delivery caused by rupture of the fetal membranes. Thus, abnormally high levels of salivary protease levels indicates elevated risk of PROM, and provides an indication for therapeutic intervention to prevent PROM-induced preterm labor.

### BRIEF DESCRIPTION OF THE DRAWINGS

FIG. 1. Data demonstrates that protease activity can be measured in saliva. Matrix metalloproteinase (MMP) activity was determined by the addition of BB-94 (MMP Inhibitor—at 25 mM) and Metalloprotease activity was detected by the addition of EDTA (2 μg/ml).

FIG. 2. MP and MMP rates in saliva samples from 10 different human volunteers. Matrix metalloproteinase (MMP) activity was determined by subtracting BB-94 (at 25 mM)—inhibitable activity from total protease activity.

FIG. 3. Inter-sample variability among 10 healthy volunteers. Sample to sample variation with individual donors were calculated by collecting saliva samples from 10 different donors and protease activity measured as in FIGS. 1 and 2.

FIG. 4. Intra-sample variability over 3 weeks in 3 individuals. Rate distribution of MMP activity range with same donor was calculated by assaying 5 different samples collected from the same donor randomly. Data indicated that the activity is fairly reproducible in individual donors.

FIG. 5. Salivary protease activities in samples collected from pregnant women. The activity was detectable and it was shown to increase as gestation progress. See Example 2.

FIG. 6. Zymogram analysis showing that salivary protease activity is predominantly MMP 9 specific. See Example 1.

FIG. 7. MMP activity is maximal in saliva samples from pPROM compared to second trimester samples: MMP activity was compared with samples collected from women who had pPROM and delivered preterm. Rate of activity from these samples were compared second trimester (gestational age matched with pPROM), with term (active labor), postpartum (term, collected within 3 hours after delivery) and non-pregnant controls. Maximum rate of activity was noticed in samples from postpartum after term delivery followed by pPROM.

FIG. 8. MMP Activity in saliva samples from pregnant women and also women with pPROM. Postpartum samples showed highest rate of activity. Active labor followed by membrane rupture, fetal and placental delivery is all prompted by MMP activity. Total protease activity was predominantly MMP activity. Salivary MMP activity rate increases in pPROM, which is well above second trimester and term active labor rates.

FIG. 9. Total, active and inhibitor-free forms of MMP 9 in saliva samples. The data was supportive of total and MMP protease activity. Maximum concentration of total and active MMP9 was seen in saliva samples from women with pPROM compared to term active labor samples. Total MMP9 was detectable in non pregnant control saliva, however active MMP9 was not detectable non these samples.

FIG. 10. Active and inhibitor free forms of MMP9 are increased in saliva samples collected from women with pPROM.

### DETAILED DESCRIPTION OF THE INVENTION

The invention provides salivary protease assays for predicting and monitoring the development and progression of preterm premature rupture of the fetal membranes (pPROM). The assays of the invention measure protease activity in saliva as a marker for protease activity in fetal membranes and in the amniotic fluid. Applicants believe that high levels of certain proteases, such as the protease MMP-9 and other MMPs, are specifically involved in matrix degradation leading to premature rupture. In addition, applicants have determined that protease levels in saliva generally increase throughout pregnancy, peaking at term and post-term. Moreover, applicants have determined that women experiencing pPROM have significantly higher salivary protease levels than women undergoing normal term deliveries, indicating that higher levels of proteases in the saliva can be used as a surrogate marker for higher levels in the fetal membranes and amniotic fluid. Higher levels of these proteases would be expected to initiate the degradation of the fetal membranes and therefore precede a rupture event. Accordingly, the invention provides a simple means for monitoring a pregnant patient's protease levels and for identifying a protease level increase that is indicative of an impending pPROM event, thus enabling interventional therapies to prevent the pPROM event. The assays of the invention may be particularly useful in monitoring women at risk for pPROM.

A programmed collagenolytic remodeling process exists in amniochorion during gestation to accommodate to the increasing uterine pressure and volume as gestation progress. This controlled collagenolytic process is mediated by MMPs each of which degrades a type-specific substrate. There are a number of regulatory mechanisms that can influence the ultimate impact of an MMP on ECM degradation. MMP activity is tightly regulated not only via control of transcription and translation but also at the post-translational level (activation of zymogen forms of MMPs) as well as at the tissue level (by specific regulators known as the tissue inhibitors of metalloproteinase—TIMP). A balanced activity between MMPs and TIMPs has been documented in tissue remodeling in several systems. In humans, there are 24 known members of the MMP family of proteins and four members of the TIMP family. The role of MMPs in human labor and PROM has been studied over the past ten years. Increased collagenolysis, a drop in the collagen content of the membranes and activation of MMPs have been documented during active labor. Several lines of evidence have documented the significance of MMPs in PROM.

As demonstrated by the studies described in Example 1, supra, saliva represents an easily accessible bodily fluid that can be repeatedly sampled to assay in vivo protease activity. A variety of protease activities are present in human saliva and these may be conveniently assayed with a generic substrate, gelatin. Inclusion of specific classes of protease inhibitors in the assays allows categorization of the activities present. The predominant metalloprotease present in saliva is MMP-9.

Total protease activity, metalloprotease activity and specific protease activities (i.e., MMP-9 activity) may be used to provide an indication of protease levels in amniochorion and fetal membranes, and thereby provide an indication of increased risk (versus normal status) of a pregnant individual developing pPROM. In addition, MMP9 protein concentration in saliva may be used to provide an indication of increased risk (or normal status) in pregnant individuals. In one embodiment, increased levels of salivary protease activity and/or MMP9 protein concentration, relative to normal levels, provides and indication of increased risk of pPROM. Normal levels (and ranges) may be determined by quantifying salivary protease activities and/or MMP9 protein concentrations for statistically significant group of pregnant individuals experiencing a normal pregnancy and term delivery.

Alternatively, in preferred embodiments, baseline protease levels are determined on an individual basis before gestation and/or during the first trimester and/or the second trimester of pregnancy. Having a precise baseline protease profile for an individual patient will enable more precise tracking of protease levels throughout gestation. The amniochorionic cavity if formed and the amniotic fluid established by 18 weeks. Accordingly, in one embodiment, baseline levels of protease activities or MMP9 protein concentrations are determined no later than the 18 week time point in order to provide a mid-second trimester baseline level. Thereafter, salivary protease protein and/or activity levels are monitored as appropriate (i.e., weekly). A significant increase in protease activity or MMP9 protein concentration, relative to the established baseline level, provides an indication of increased risk of the individual developing pPROM.

In one embodiment, the salivary levels of MMP9 protein and/or protease activities are determined on a weekly basis from the earliest point in the first trimester through term. Baseline levels are thus established during the first and second trimester. Significant and/or sharp elevations in the level of protease activity or MMP9 concentration during the second or third trimester provide an indication of increased risk of preterm delivery induced by PROM.

Salivary MMP9 protein concentration may be determined using methods known in the art. In preferred embodiments, MMP9 protein concentration is determined by immunoassay, typically utilizing an MMP9-specific antibody reagent. In one embodiment, the immunoassay is an ELISA. However, as will be appreciated by those skilled in the art, many other immunoassay formats may be used to determine MMP9 protein concentration, including without limitation, immunohistochemical assays, Western blot, radioimmunoassay, immunometric assays, competitive immunoassays, fluorescent immunosorbent assays, chemical-linked immunosorbent assays, immunoblotting, and the like. An example of a suitable immunoassay is described in Example 3, infra.

In a specific embodiment, a method for determining whether a pregnant individual is at increased risk of preterm delivery induced by premature rupture of the fetal membranes is provided, and comprises obtaining a saliva sample from the individual, determining the concentration of MMP9 protein in the saliva sample, and comparing the MMP9 protein concentration in the saliva sample to a normal salivary MMP9 protein concentration. A significant increase in the individual's MMP9 protein concentration relative to the normal MMP9 protein concentration provides an indication of increased risk. Typically, the concentration of MMP9 protein is determined using a standard immunoassay, such as an ELISA, although any immunoassay or other method for quantifying MMP9 protein may be used.

In a related embodiment, a method for determining whether a pregnant individual is at increased risk of preterm delivery induced by premature rupture of the fetal membranes is provided, and comprises obtaining a saliva sample from the individual, determining the concentration of MMP9 protein in the saliva sample, and comparing the MMP9 protein concentration in the saliva sample to a previously-determined baseline salivary MMP9 protein concentration in that individual. A significant increase in the individual's MMP9 protein concentration relative to the baseline MMP9 protein concentration for that individual provides an indication of increased risk. Baseline salivary MMP9 protein concentrations may be determined during the first and/or second trimester of pregnancy, and/or prior to pregnancy. When determining baseline concentrations during pregnancy, it is preferred that multiple measurements are taken. In one embodiment, baseline salivary MMP9 concentration is determined by measuring salivary MMP9 protein concentration in the individual between about 6 and about 18 weeks gestation, or between about 12 and about 18 weeks gestation. Salivary samples are then monitored for elevated MMP9 protein levels, typically after 18 weeks gestation. Monitoring on a weekly basis is preferred. However, in individuals with other risk factors (i.e., prior PROM, pPROM or premature delivery history), it may be preferable to monitor MMP9 protein concentrations on a more frequent basis after 18 weeks gestation.

A significant increase in the individual's MMP9 protein concentration relative to the normal MMP9 protein concentration may be a statistically-significant increase. Alternatively, a significant increase in the individual's MMP9 protein concentration relative to the normal MMP9 protein concentration may be at least a two-fold, at least a three-fold, at least a four-fold, or at least a five-fold increase.

In a related aspect, MMP9 biological activity may be measured, as an alternative to measuring MMP9 protein concentration. MMP9 activity assays are known. One commercially available assay is the Biotrak MMP9 immuno-capture activity assay (Amersham Pharmacia Biotech Inc, Piscataway, N.J., USA).

In another aspect of the invention, total salivary proteinase activity may be used to provide an indication of increased risk of preterm delivery induced by premature rupture of the fetal membranes. Examples of salivary proteinase assays are provided in the Examples which follow. In a specific embodiment, total proteolytic activity in saliva samples is measured by fluorometry using the generic substrate DQ-gelatin (see Example 1, infra).

In another aspect of the invention, metalloproteinase (MP) and/or matrix metalloproteinase (MMP) activities are used to provide an indication of increased risk of preterm delivery induced by premature rupture of the fetal membranes. As the results in Examples 1 and 2 show, the MP activity in saliva is predominantly due to MMP activity. Thus, in one embodiment, total MP activity is used. In a related embodiment, total MMP activity is used.

### EXAMPLES

### Example 1

### Proteinase Saliva Assay

### Materials and Methods:

Human saliva collection: Saliva samples (˜2 ml) were collected from healthy volunteers using a commercially-available Salivette™ T device (Sarstedt, Newton, N.C., USA). Samples were collected after subjects had fasted for at least one hour. Subjects were instructed to rinse their mouths with water before chewing the Salivette wad for about 1 minute so as to saturate it with oral secretions, principally saliva. The samples were either used fresh or stored at −20° C., a process demonstrated not to affect activity.

Zymography: 20 μl of each saliva sample was mixed with 5 μl of a sample buffer containing 250 mM Tris pH 6.8, 25% glycerol, 10% (w/v) SDS, 0.01% bromophenol blue and loaded onto 10% SDS-PAGE gels containing 0.25% porcine gelatin (Sigma). Molecular weight markers (Biowhittaker Molecular Applications, Rockland, Me., USA) and a positive control sample of conditioned medium from the human fibrosarcoma cell line HT-1080 were also included on the gel. Following electrophoresis, the gels were washed twice for 15 mins each in 2.5% Triton X-100 and then incubated overnight at 37° C. in a substrate buffer containing 50 mM Tris-HCl, pH 7.6 and 10 mM CaCl₂ in the absence or presence of proteinase inhibitors EDTA (30 mM), BB-94 (20 μM), BMS-275291 (20 μM) or B428 (20 μM). Following incubation, the gels were stained with a solution of 0.5% (w/v) Coomassie Blue R250 prepared in 50% methanol: 10% acetic acid. Once stained, the gels were briefly de-stained in the same solution without the Coomassie Blue and then stored in distilled water. Areas of gelatinolytic activity appeared cleared against the blue background of the coomassie-stained undigested gelatin.

Western Blotting: 30 μl each of the saliva samples were mixed with a sample buffer as before with the addition of 0.15 M β-mercaptoethanol and boiled for 10 minutes prior to loading onto 10% SDS-PAGE gels. Following electrophoresis, the separated proteins were transferred to nitrocellulose at 100V for 2 hrs. The nitrocellulose was blocked with 10% milk in tris-buffered saline (TBS: 0.15 M NaCl, 0.01 M Tris-HCl, pH 7.5) for 1 hr at room temperature and then incubated overnight at 4° C. with anti-human MMP-9 antibody (Ab-1: Calbiochem, San Diego, Calif., USA) diluted 1:1000 in blocking solution. After multiple washes in TBS containing 0.05% Tween-20 (TBS-T), the blots were incubated for 30 mins at room temperature with biotinylated anti-mouse antibody (Vector Laboratories, Burlingame, Calif.) diluted 1:15,000 in blocking solution. After another series of washes in TBS-T, the blots were probed for 30 mins at room temp. with peroxidase-conjugated streptavidin (Vector) diluted 1:15,000 in blocking solution. After a final series of TBS-T washes, the blots were analyzed by chemiluminescence using Western Lightening reagents (Perkin-Elmer, Boston, Mass.), according to manufacturer's instructions and exposure to film (Fuji Super RX, Fuji, Stamford Conn.).

MMP-9 activity assay The commercially available Biotrak MMP9 immuno-capture activity assay (Amersham Pharmacia Biotech Inc, Piscataway, N.J., USA) was used to determine levels of active MMP-9 in saliva samples according to manufacturer's instructions. Samples were assayed without additional activation to assess the levels of endogenously active MMP9 although it should be noted that the assay is reported by the manufacturer to have some cross-reactivity with pro-MMP9.

Amylase assay: In order to normalize pre- and post-treatment saliva samples, total amylase activity was measured using the Enz-Chek amylase assay kit (Molecular Probes Inc, Eugene, Oreg.) according to manufacturer's instructions.

Proteinase activity assay: Total proteolytic activity in saliva samples was measured by fluorometry using the generic substrate DQ-gelatin (Molecular Probes Inc). An aliquot (usually 50 μl per 160 μl total assay volume) of each sample was mixed with substrate (10 μg/ml) in tricine buffer (50 mM Tricine, pH 7.4; 200 mM NaCl; 10 mM CaCl₂; 0.05 mM ZnSO₄; 0.005% Brij 35) and incubated at 37° C. An inhibitor cocktail comprising of inhibitors of serine, cysteine and aspartyl proteinases (‘Complete—EDTA free’, Roche Biochemicals, San Diego, Calif.) was included in the reactions for all the human saliva samples. Fluorometric measurements (excitation at 485 nm and emission at 538 nm) recorded every 10-20 minutes for 2 hours. These values were used to calculate an initial rate of enzyme activity (Fluorescence units/minute) which, for the murine samples, was then normalized for amylase concentration. To measure non-metalloproteinase activity, EDTA at a concentration of 30 mM was included in the assay mixture. Metalloproteinase (MP) activity was then determined as total less the EDTA-inhibited activity. Similarly, matrix metalloproteinase (MMP) activity, defined as the BB-94-inhibitible activity, was determined by subtracting the activity in the presence of BB-94 (25 μM) from total proteinase activity. To measure non-serine proteinase activity, aprotinin (2 mg/ml) and leupeptin (100 mM) were included in the assay.

LC-MS analysis: Chromatographic separation of the saliva samples was performed using a Surveyor HPLC in tandem with a ThermoFinnigan (San Jose, Calif.) TSQ Quantum triple quadrupole mass spectrometer equipped with a standard electrospray ionization source outfitted with a 100μ I.D. deactivated fused silica capillary. All chromatographic separations were performed on a Phenomenex (Torrance, Calif.) Prodigy HPLC column, ODS (3) 100A 150×2 mm 5 microns. The mobile phase component for the assays consisted of A=5% Acetonitrile: 2% Ethanol: 93% MilliQ Water: 0.3% Formic Acid and B=80% Acetonitrile: 10% Ethanol: 10% MilliQ Water: 0.3% Formic Acid. The gradient for all analyses began at 10% B, gradually changing the composition of the mobile phase to 95%-100% B and returning to the starting composition. The flow rate was 200 uL/min. Nitrogen was used for both the sheath and auxiliary gas, which were set to 45 and 24 (arbitrary units), respectively. Mass spectrometer operation was in the positive ion mode and the ion transfer tube was operated at 35V and 280° C. (for BMS 275291) or 320° C. (for B428 and BB94). The tube lens voltage was set to 112V. Source CID was used at 15V. The mass spectrometer was used in the selected reaction monitoring mode. Ions were collisionally activated with argon at an indicated pressure of 1.5 mT. The mass-spectral resolution was set to a peak width of 0.7 u for both precursor and product ions. Mass transitions at the specified collision energy (m/z 609.3→195.1, 42 eV), (m/z 302.7→159.1, 24 eV), (m/z 605.4→461.2, 25 eV) and (m/z 478.2→267.1, 20 eV) were monitored for reserpine (used as an internal standard in all analyses), B428, BMS-275291 and BB94, respectively. The scan width for product ions was 1.0 u and the cycle time for each ion was 0.5 seconds. The electron multiplier gain was set to 2×10⁶. Data were acquired in profile mode. Xcalibur software, version 1.3, from ThermoFinnigan was used on a Dell Optiplex GX240. The proteins contained in the saliva samples were precipitated out using a volume of acetonitrile equal to 5 times the volume of the sample. The acetonitrile eluant was taken to dryness using a Savant Speed Vac and brought to 100 uL using mobile phase A. The BMS-275291 was not precipitated and 50 uL of these samples and standards were used for a reduction/alkylation reaction.

This was initiated by the addition of 25 μl 1.0M N-ethylmorpholine, 10 μl TCEP and 50 μl of the internal standard reserpine. The mixture was heated to 60° C. for 30 min using a standard heatblock, after which 10 μl of 4-vinylpyridine was added. After a second 30-min incubation at 60° C., the sample was ready for analysis. The calibration ranges were 1-1132 ng/ml for B428, 28-360 ng/ml for BB-94, and 25-499 ng/ml for BMS-275291.

### Results:

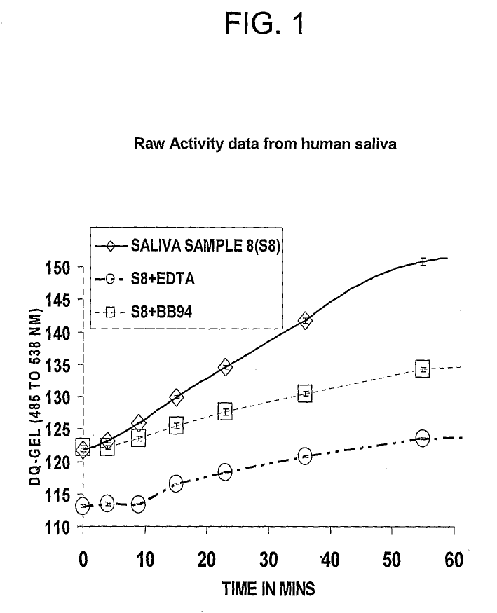

Human saliva samples were collected using a Salivette™ device in which the test subject chews a provided synthetic wad for 1 minute to fully saturate it with saliva after which the saliva is extracted by a brief centrifugation. Proteinase activity was then assayed using DQ-gelatin, a commercially available gelatin substrate heavily labeled with fluorescein such that it is minimally fluorescent due to self-quenching until cleaved by proteinase. Addition of 50 μl of human saliva to this substrate at neutral pH in the presence of a mix of general inhibitors of serine, cysteine and aspartyl proteinases resulted in a measurable time-dependent increase in fluorescence attributed to cleavage of the DQ-gelatin by proteinases including metalloproteinases (MP). The initial increase in fluorescence was approximately linear with time usually for 30 min or longer (FIG. 1) indicating that cleavage of the substrate exhibits first-order kinetics and from which proteinase activity, expressed in arbitrary fluorescence units, could be calculated. For each saliva sample, proteinase activity was also measured in the presence of either EDTA, a general divalent cation chelator or BB-94, an hydroxamic acid-based synthetic inhibitor. This allowed a determination of metalloproteinase (MP) activity identified as EDTA-inhibitable and matrix metalloproteinase (MMP) activity defined as BB-94-inhibitable (FIG. 1).

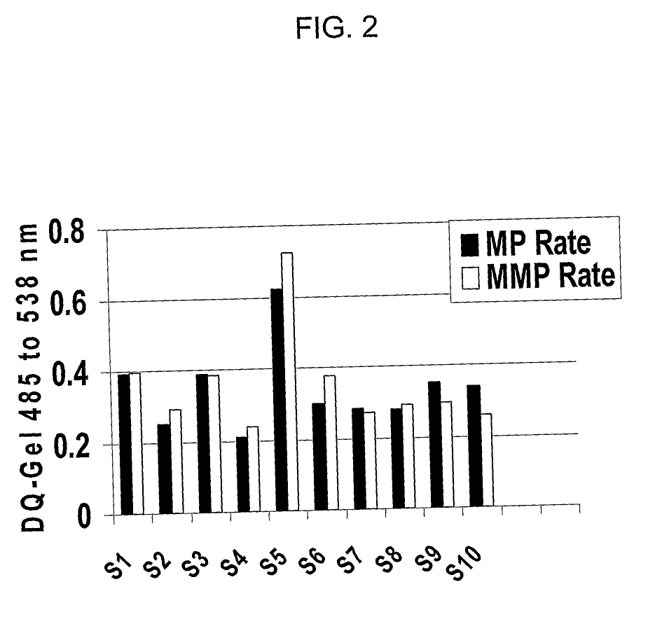

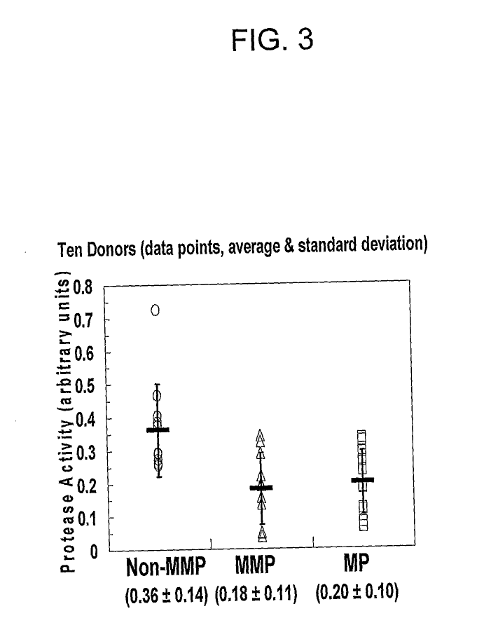

In the human samples tested, the MP and MMP activities were generally equivalent (FIGS. 2 and 3) indicating that the metalloproteinases in saliva that cleave DQ-gelatin are predominantly MMPs. The MP and MMP activities of saliva collected using the Salivette™ were comparable to those of saliva collected directly from the same subject and cleared by centrifugation. BB-94 was originally developed as a broad-spectrum inhibitor of matrix metalloproteinases (MMPs), however it has also been shown to have activity against sheddases, proteinases of the ADAM family that share homology with MMPs. A second synthetic inhibitor of MMPs, BMS-275291, designed to avoid sheddase inhibition gave results comparable to those with BB-94 indicating that, in human saliva samples, the metalloproteinase activity can be ascribed to the MMP sub-class. To facilitate the planning of future saliva collections, the effect of storage at −20° C. on the MP or MMP activity of saliva samples was also assessed, and no significant difference between measured MP or MMP rates for fresh samples versus those stored at −20° C. was observed. However, multiple rounds of freezing and thawing did lead to appreciable changes, and so this was avoided.

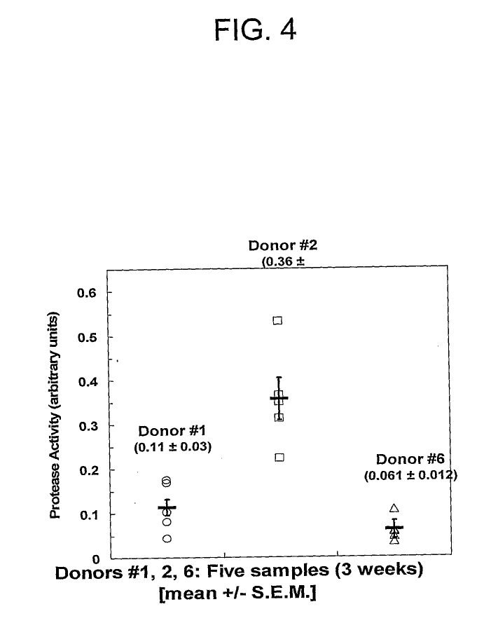

The levels of total gelatinase as well as MP and MMP activity among a group of eleven healthy donors (fourteen total samples), both male and female in the age range 25-50 years old exhibit significant subject-to-subject variability (61%) with 2 of the 11 donors having no appreciable MMP activity (FIG. 3). This variation did not correlate with gender or age of the donors. We also assayed 5 samples from each of three individuals collected over a three week period to assess the intra-donor variability (FIG. 4). Under these conditions, the variability within any one individual was relatively small, with a maximum of 22%. Overall, the inter-assay precision was excellent with a coefficient of variation (c.v.) of 6% while the intra-assay c.v. was 10%.

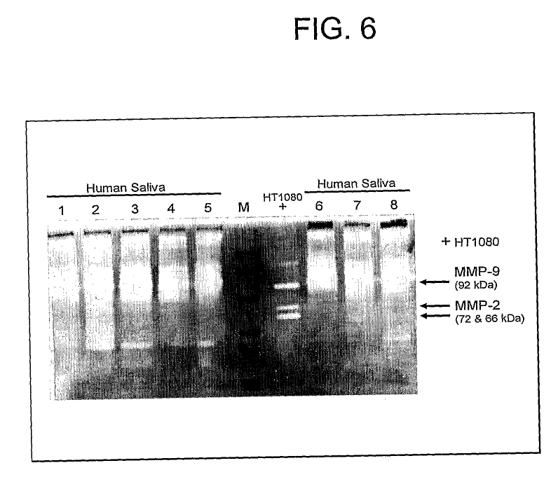

Saliva samples were also tested for the presence of gelatin-degrading MMPs. Samples were exposed to gelatin zymography to visualize the proteinase activity acting on a gelatin substrate. Based on molecular weight, it is apparent that the major gelatin-degrading activity present in human saliva is MMP-9 (FIG. 6). The majority of the gelatinase activity has an apparent size of ˜120 kDa, which corresponds to the MMP-9/lipocalin complex previously reported in saliva. The presence of MMP-9 was confirmed by western blot analysis of the saliva samples using an antibody specific for human pro-MMP9. Finally, the saliva samples were assessed using a commercially-available immuno-capture activity assay specifically for human MMP-9. This assay has previously been used to demonstrate increased MMP-9 activity in saliva samples from patients with Sjogrens syndrome. Using this method, active MMP-9 was detected in all the samples tested ranging in concentration from 0.19 to 0.48 ng/ml. When the data from the fluorometric activity assay was compared with that from the MMP-9 specific activity assay, a relatively high degree of correlation could be observed. One sample that showed a high MMP-9 level by the immuno-capture assay was one that showed minimal MP or MMP activity by the fluorometric assay, which may reflect some detection of the pro-form of MMP-9 in the immuno-capture assay. However, taken together, these data point to MMP-9 as being the major metalloproteinase source of gelatinolytic activity in the human saliva samples.

### Example 2

### Salivary Protease Activity in Human Pregnancy

### Materials and Methods

Saliva samples were assayed for MMP activity from women in the second trimester of pregnancy, term (active labor and postpartum) and also from women who had PPROM and had spontaneous preterm deliveries. All saliva samples collected from pregnant and non-pregnant women exhibited proteolytic activity, predominantly MMP activity under the conditions for assay. Samples were analyzed using the DQ-Gelatin based MMP activity assay (see Example 1).

### Results:

Saliva samples collected during pregnancy exhibited MP activity including MMP activity, defined as the proteolytic activity inhibitable by BB94. As in the saliva samples from non-pregnant individuals (see FIG. 2), the MMP activity constitutes most of the MP activity detected using this assay. The BB-94 inhibitable activity may also include some ADAMs activities, since this broad-spectrum MMPI has been shown to also inhibit the activities of at least some of the ADAMs proteinases.

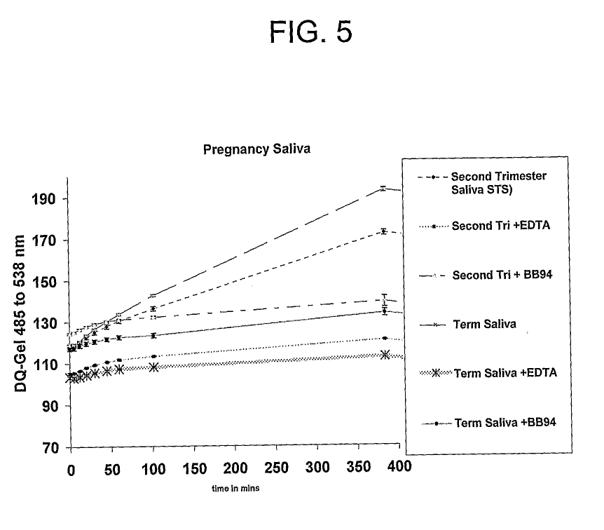

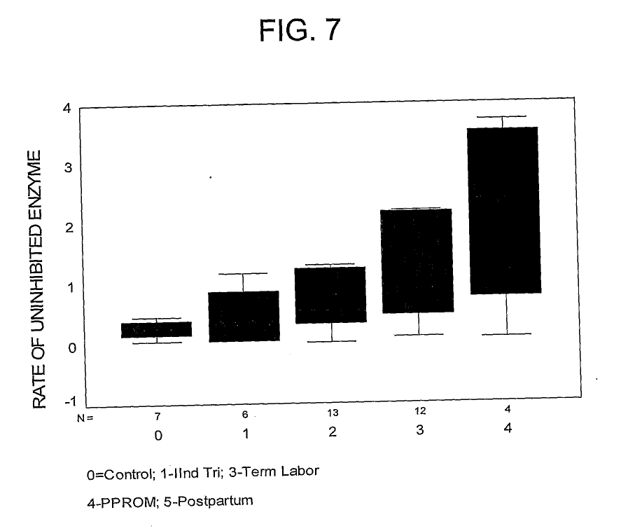

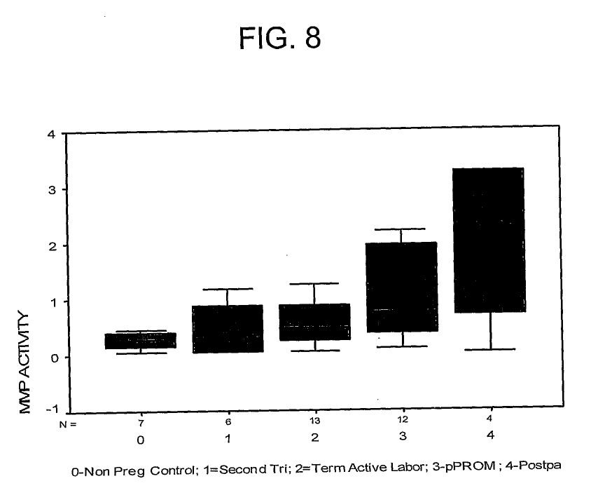

In these studies of saliva samples collected during pregnancy, both MP and MMP rates were higher in term samples compared to preterm (second trimester) samples (FIG. 5). Additionally, salivary MMP activity is higher in pregnant women compared to non-pregnant control (FIGS. 7 and 8). The activity increases during pregnancy and it is maximum at term during labor. MMP activity was minimal in non-pregnant controls (mean ±std: 0.25±0.15) compared to activity in saliva from pregnant women. Samples collected from women with pPROM had the highest activity (2.52±3.7) followed by those from women after normal term delivery (2.1±1.6) (FIG. 7, 8). Salivary MMP activity is low at second trimester (0.5±0.5) and increases at term during active labor (1.03±1.2) samples.

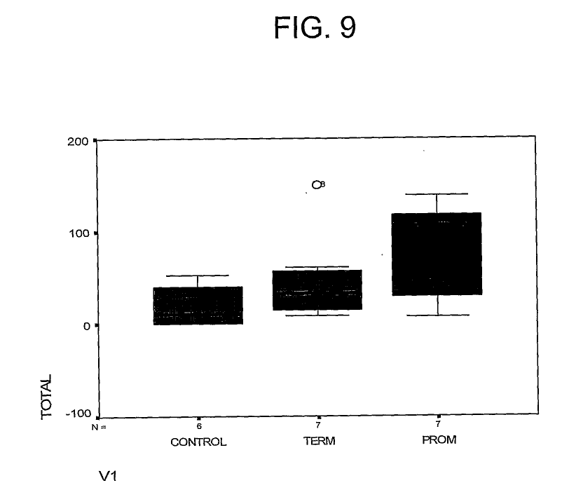

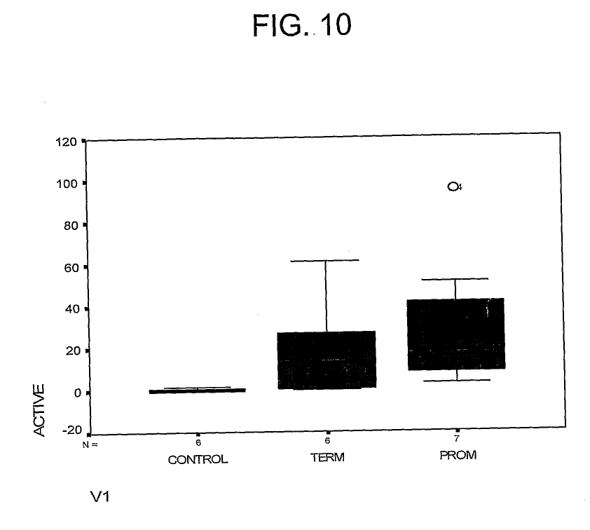

A two-fold increase in MMP activity was seen in pPROM compared to term labor (FIGS. 7-10). These results indicate that salivary MMP activity is a systemic response of membrane level MMP activity which is documented to change during pregnancy and increase at term. A summary of protease activity, MMP activity and MP activity are listed in Table I below.

TABLE I
Rate of enzyme activity in saliva samples

| Group (n) | | Mean ± Std |
|---|---|---|
| Control (7) | Total Rate | 0.25 ± 0.15 |
| | MMP Rate | 0.27 ± 0.15 |
| Second Trimester | Total Rate | 0.48 ± 0.46 |
| (6) | MMP Rate | 0.47 ± 0.46 |
| Term Active Labor | Total Rate | 0.64 ± 0.4 |
| (13) | MMP Rate | 0.9 ± 1.24 |
| pPROM (12) | Total Rate | 3.3 ± 4.4 |
| | MMP Rate | 2.42 ± 3.0 |

### Example 3

### Solid Phase Immunoassay for MMP Protein Concentration in Saliva

### Materials and Methods:

Multiple bead assays are solid phase sandwich immunoassays, which are designed to be analyzed with a Luminex instrument. This technology is based on advances in microsphere technology and has led to the development of flow cytometric assays which allow the simultaneous detection of up to 100 analytes in a single microtiter well. The MMP assay, which was utilized in this study, is designed in a conventional capture sandwich immunoassay format. MMP specific antibodies are covalently coupled to color coded 5.6 μm beads. The antibody-coupled beads (R&D System, Minneapolis, Minn.) were incubated with known (standards; a broad range 8-point standard curve covering the expected range of concentrations were used) and unknown (saliva sample) amounts of MMP. After a series of brief washes to remove unbound protein, a biotinylated detection antibody specific for a different epitope on the MMP were added to the beads. The reaction mixture was detected by the addition of streptavidin-phycoerythrin, which will bind to the biotinylated detection antibodies using flow-based Luminex® system, which will identify and quantify each analyte of interest. If it is anticipated that the MMP levels are higher than the highest standard, samples were diluted with assay buffer (1:1). Luminex® Data Collector software, version 1.7, (Luminex® Corporation, Austin Tex.), Bio-Plex Manager data reduction software (Bio-Rad Laboratories, Hercules, Calif.) and slidewrite Plus data analysis software for windows, version 5.0 (Advanced Graphics Software Inc., Encinitas, Calif.) were used for data analysis. Assay buffer along with two known calibration controls were run along with the samples. The median fluorescent intensity data were collected utilizing the Luminex® Data Collector Software and data were imported into the Bio-Plex Manager software in order to perform data reduction. Standard curves were generated and MMP concentrations were interpolated for each sample utilizing a 5-parameter curve fit equation.

### Results:

Multiplex assay was performed to measure the concentrations of various salivary MMPs: collagenases (MMP1 and MMP 13), stromelysin (MMP3), gelatinases (MMP2 and 9), Matrilysin (MMP 7) and macrophage elastase (MMP 12) (See Table II). No statistically significant differences in the individual MMP concentrations were detected during pregnancy compared to non pregnant controls except for MMP7. MMP7 levels were higher in non-pregnant women (283 pg/ml) compared to samples at all stages of pregnancy. Similarly a non-significant drop in the amount of MMP9 was observed during pregnancy. MMP2 and MMP3 concentrations were also lower in the second trimester (131.9 and 8.6 pg/ml respectively) compared to non pregnant control (166.6 and 15.9 pg/ml), however doubled at term (213.5 and 15.5 pg/ml) to levels similar to those from non pregnant females. The concentrations of both MMP9 and MMP13 also appear to increase in postpartum samples compared to term labor although these concentrations were not significantly different compared to those of non-pregnant controls.

TABLE II
Concentration of MMPs in saliva as detected by multiplex assay

| | Non Pregnant | | | | |
|---|---|---|---|---|---|
| MMPs | Controls | IInd Trimester | Term Labor | Postpartum | pPROM |
| MMP 1 | 16.34 ± 1.6 | 17.27 ± 0.9 | 16.16 ± 1.8 | 16.5 ± 0.1 | 17.3 ± 1.1 |
| MMP 2 | 166.6 ± 37 | 131.9 ± 11.6 | 213.5 ± 215 | 196.1 ± 70 | 141.3 ± 26.5 |
| MMP 3 | 15.89 ± 8 | 8.6 ± 1.06 | 15.49 ± 17.8 | 9.4 ± 1.8 | 20.75 ± 14.4 |
| MMP 7 | 283.0 ± 194.6 | 48.32 ± 7.4 | 54.18 ± 22.4 | 43.7 ± 5.4 | 67.12 ± 49.8 |
| MMP 9 | 8366.6 ± 6148 | 4460.6 ± 3327 | 4068.9 ± 3767 | 8383 ± 8906 | 14887 ± 12920 |
| MMP12 | 9.6 ± 1.8 | 8.4 ± 0.5 | 8.2 ± 1.5 | 8.85 ± 0.5 | 7.9 ± 0.5 |
| MMP 13 | 41.3 ± 3.5 | 42.3 ± 2.3 | 43.2 ± 6 | 53.03 ± 29.1 | 39.3 ± 5.2 |

All units: pg/ml

The concentrations of MMP9 protein, as measured by Luminex, were highest in pPROM samples (14887 pg/ml) compared to term (4068.9 pg/ml; p=0.01), postpartum (8383 pg/m; p=0.4), or in any other groups. MMP9 protein was the only MMP that was increased in saliva from pPROM patients. This was further consistent with gelatin zymography results and an activity assay selective for MMP9 (see Examples 1 and 2, supra). Zymography demonstrated MMP9 specific bands (92 kDa) in almost all the samples tested with active forms (83 KDa) and little or no MMP2 specific bands (72 kDa). Controls gels treated with EDTA showed no bands. The MMP9 activity assay documented higher concentration of active inhibitor free MMP9 in saliva from women with pPROM (45.3±33.8 pg/ml) compared to term active labor samples (18.9±23.9 pg/ml; p=0.04) or postpartum samples (31.2±41.2 pg/ml; p=0.2)). Saliva from non-pregnant controls showed less than 0.3 pg/ml of active MMP9.

All publications, patents, and patent applications cited in this specification are herein incorporated by reference as if each individual publication or patent application were specifically and individually indicated to be incorporated by reference.

The present invention is not to be limited in scope by the embodiments disclosed herein, which are intended as single illustrations of individual aspects of the invention, and any which are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

### CITATION OF RELATED LITERATURE

1. Parry S, Strauss J F 3rd. Premature rupture of the fetal membranes. N Engl J Med 1998; 338:663-70.
2. Guyer B, MacDorman M F, Martin J A, Peters K D, Strobino D M. Annual summary of vital statistics-997. Pediatrics 1998; 102:1333-49.
3. Savitz D A, Blackmore C A, Thorp J M. Epidemiologic characteristics of preterm delivery: etiologic heterogeneity. Am J Obstet Gynecol 1991; 164:467-71.
4. French J I, McGregor J A. The pathobiology of premature rupture of membranes. Semin Perinatol 1996; 20:344-68.
5. Skinner S J, Campos G A, Liggins G C. Collagen content of human amniotic membranes: effect of gestation length and premature rupture. Obstet Gynecol 1981; 57:487-9.
6. Gomez R, Romero R, Edwin S S, David C. Pathogenesis of preterm labor and preterm premature rupture of membranes associated with intraamniotic infection. Infect Dis Clin North Am 1997; 11:135-76.
7. Fortunato S J, Menon R, Lombardi S J. Coliagenolytic enzymes (gelatinases) and their inhibitors in human amniochorionic membrane. Am J. Obstet Gynecol 1997; 177:731-741.
8. Draper D, McGregor J, Hall J et al. Elevated protease activities in human amnion and chorion correlate with preterm premature rupture of membranes. Am J Obstet Gynecol 1995; 173:1506-12.
9. So T. The role of matrix metalloproteinases for premature rupture of the membranes. Nippon Sanka Fujinka Gakkai Zasshi 1993; 45:227-33.
10. Woesner F J. Matrix metalloproteinases and their inhibitors in connective tissue remodeling. FASEB J 1991; 5:2145-54.
11. Vadillo-Ortega F, Hernandez A, Gonzalez-Avila G, Bermejo L, Iwata K, Strauss J F 3rd. Increased matrix metalloproteinase activity and reduced tissue inhibitor of metalloproteinases-1 levels in amniotic fluids from pregnancies complicated by premature rupture of membranes. Am J Obstet Gynecol. 1996; 174:1371-6.
12. Fortunato S J, Menon R, Lombardi S J. MMP/TIMP imbalance in amniotic fluid during PPROM: an indirect support for endogenous pathway to membrane rupture. J Perinat Med 1999; 27:362-8.
13. Menon R, Fortunato S J. Preterm Premature Rupture of the Membranes: A network of MMPs and Apoptosis. J Soc Gyn Invest, 2004.
14. Fortunato S J, Menon R, Lombardi S J. Amniochorion gelatinase-gelatinase inhibitor imbalance in vitro: a possible infectious pathway to rupture. Obstet Gynecol 2000; 95:240-4.
15. Athayde N, Edwin S S, Romero R, Gomez R, Maymon E, Pacora P, Menon R. A role for matrix metalloproteinase-9 in spontaneous rupture of the fetal membranes. Am J Obstet Gynecol 1998; 179:1248-53.
16. Athayde N, Romero R, Gomez R, Maymon E, Pacora P, Mazor M, Yoon B H, Fortunato S, Menon R, Ghezzi F, Edwin S S. Matrix metalloproteinases-9 in preterm and term human parturition. J Matern Fetal Med 1999; 8:213-9.
17. Fortunato S J, Menon R, Lombardi S J, LaFleur B. Interleukin-10 inhibition of gelatinases in fetal membranes: therapeutic implications in preterm premature rupture of membranes. Obstet Gynecol 2001; 98:284-8.
18. Ingman, T., Tervahartiala, T., Ding, Y., Tschesche, H., Haerian, A., Kinane, D. F., Konttinen, Y. T., and Sorsa, T. Matrix metalloproteinases and their inhibitors in gingival crevicular fluid and saliva of periodontitis patients. J Clin Periodontol 1996; 23:1127-1132.
19. Overall, C. M., Sodek, J., McCulloch, C. A. G., and Birek, P. Evidence for polymorphonuclear leukocyte collagenase and 92-kilodalton gelatinase in gingival crevicular fluid. Infect Immun 1991; 59:4687-4692.
20. Fingleton B, Menon R, Fortunato S J, Carter K J, Overstreet P D, Hachey D L, Matrisian L M, Mcintyre J O. Proteinase Activity in Human and Murine Saliva as a Biomarker for Proteinase Inhibitor Efficacy. Clin Cancer Res 2004 10: 7865-7874.
21. Vadillo-Ortega F, Gonzalez-Avila G, Furth E E, Lei H, Muschel R J, Stetler-Stevenson W G, Strauss J F. 92-kd type IV collagenase (matrix metalloproteinase-9) activity in human amniochorion increases with labor. Am Pathol 1995; 146:148-156.
22. Vadillo-Ortega F, Hernandez A, Gonzalez-Avila G, Bermejo L, Iwata K, Strauss J F 3rd. Increased matrix metalloproteinase activity and reduced tissue inhibitor of metalloproteinases-1 levels in amniotic fluids from pregnancies complicated by premature rupture of membranes. Am J Obstet Gynecol. 1996; 174:1371-6.
23. Fortunato S J, Menon R, Lombardi S J. Collagenolytic enzymes (gelatinases) and their inhibitors in human amniochorionic membranes). Am J Obstet Gynecol 1997; 77:731-41.
24. McLaren J, Taylor D J, Bell S C. Increased concentration of pro-matrix metalloproteinase 9 in term fetal membranes overlying the cervix before labor: implications for membrane remodeling and rupture. Am J Obstet Gynecol 2000; 182:409-16.
25. Uchide K; Ueno H; Inoue M; Sakai A; Fujimoto N; Okada Y. Matrix metalloproteinase-9 and tensile strength of fetal membranes in uncomplicated labor. Obstet Gynecol 2000; 95:851-5.

What is claimed is:

1. A method for determining whether a pregnant individual is at increased risk of preterm delivery induced by premature rupture of the fetal membranes, comprising:
(a) obtaining a saliva sample from the individual;
(b) determining the concentration of MMP9 protein in the saliva sample; and
(c) comparing the MMP9 protein concentration in the saliva sample to a normal salivary MMP9 protein concentration;
wherein a significant increase in the individual's salivary MMP9 protein concentration relative to the normal salivary MMP9 protein concentration provides an indication of increased risk.

2. The method of claim 1, wherein the concentration of MMP9 protein is determined using an immunoassay.

3. The method of claim 2, wherein the immunoassay is an ELISA.

4. The method of any one of claims 1-3, wherein a significant increase in the individual's salivary MMP9 protein concentration relative to the normal salivary MMP9 protein concentration is a statistically-significant increase.

5. The method of any one of claims 1-3, wherein a significant increase in the individual's salivary MMP9 protein concentration relative to the normal salivary MMP9 protein concentration is at least a two-fold increase.

6. The method of any one of claims 1-3, wherein a significant increase in the individual's salivary MMP9 protein concentration relative to the normal salivary MMP9 protein concentration is at least a three-fold increase.

7. A method for determining whether a pregnant individual is at increased risk of preterm delivery induced by premature rupture of the fetal membranes, comprising:
(a) obtaining a saliva sample from the individual;
(b) determining the concentration of MMP9 protein in the saliva sample; and
(c) comparing the MMP9 protein concentration in the saliva sample to a previously-determined baseline salivary MMP9 protein concentration in that individual;
wherein a significant increase in the individual's salivary MMP9 protein concentration relative to the baseline salivary MMP9 protein concentration provides an indication of increased risk.

8. The method of claim 7, wherein the baseline salivary MMP9 concentration is determined by measuring salivary MMP9 protein concentration in the individual between 6 and 18 weeks gestation, and the saliva sample of (a) is obtained after 18 weeks gestation.

9. The method of claim 7, wherein the baseline salivary MMP9 concentration is determined by measuring salivary MMP9 protein concentration in the individual between 12 and 18 weeks gestation, and the saliva sample of (a) is obtained after 18 weeks gestation.

10. The method of any one of claims 7-9, wherein the concentration of MMP9 protein is determined using an immunoassay.

11. The method of claim 10, wherein the immunoassay is an ELISA.

12. The method of any one of claims 7-11, wherein a significant increase in the individual's salivary MMP9 protein concentration relative to the individual's baseline salivary MMP9 protein concentration is a statistically-significant increase.

13. The method of any one of claims 7-11, wherein a significant increase in the individual's salivary MMP9 protein concentration relative to the individual's baseline salivary MMP9 protein concentration is at least a two-fold increase.

14. The method of any one of claims 7-11, wherein a significant increase in the individual's salivary MMP9 protein concentration relative to the individual's baseline salivary MMP9 protein concentration is at least a three-fold increase.

15. A method for determining whether a pregnant individual is at increased risk of preterm delivery induced by premature rupture of the fetal membranes, comprising:
(a) obtaining a saliva sample from the individual;
(b) measuring the MMP9 activity in the saliva sample; and
(c) comparing the MMP9 activity in the saliva sample to a normal salivary MMP9 activity;
wherein a significant increase in the individual's salivary MMP9 activity relative to the normal salivary MMP9 activity provides an indication of increased risk.

16. The method of claim 15, wherein a significant increase in the individual's salivary MMP9 activity relative to the normal salivary MMP9 activity is a statistically-significant increase.

17. The method of claim 15, wherein a significant increase in the individual's salivary MMP9 activity relative to the normal salivary MMP9 activity is at least a two-fold increase.

18. The method of claim 15, wherein a significant increase in the individual's salivary MMP9 activity relative to the normal salivary MMP9 activity is at least a three-fold increase.

19. A method for determining whether a pregnant individual is at increased risk of preterm delivery induced by premature rupture of the fetal membranes, comprising:
(a) obtaining a saliva sample from the individual;
(b) measuring the MMP9 activity in the saliva sample; and
(c) comparing the MMP9 activity in the saliva sample to a previously-determined baseline salivary MMP9 activity in that individual;
wherein a significant increase in the individual's salivary MMP9 activity relative to the baseline salivary MMP9 activity provides an indication of increased risk.

20. The method of claim 19, wherein the baseline salivary MMP9 activity is determined by measuring salivary MMP9 activity in the individual between 6 and 18 weeks gestation, and the saliva sample of (a) is obtained after 18 weeks gestation.

21. The method of claim 19, wherein the baseline salivary MMP9 activity is determined by measuring salivary MMP9 activity in the individual between 12 and 18 weeks gestation, and the saliva sample of (a) is obtained after 18 weeks gestation.

22. The method of any one of claims 19-21, wherein a significant increase in the individual's salivary MMP9 activity relative to the individual's baseline salivary MMP9 activity is a statistically-significant increase.

23. The method of any one of claims 19-21, wherein a significant increase in the individual's salivary MMP9 activity relative to the individual's baseline salivary MMP9 activity is at least a two-fold increase.

24. The method of any one of claims 19-21, wherein a significant increase in the individual's salivary MMP9 activity relative to the individual's baseline salivary MMP9 activity is at least a three-fold increase.

25. A method for determining whether a pregnant individual is at increased risk of preterm delivery induced by premature rupture of the fetal membranes, comprising:
(a) obtaining a saliva sample from the individual;
(b) measuring the total MP or MMP activity in the saliva sample; and
(c) comparing the total MP or MMP activity in the saliva sample to a normal salivary MP or MMP activity;
wherein a significant increase in the individual's salivary MP or MMP activity relative to the normal salivary MP or MMP activity provides an indication of increased risk.

26. A method for determining whether a pregnant individual is at increased risk of preterm delivery induced by premature rupture of the fetal membranes, comprising:
(a) obtaining a saliva sample from the individual;
(b) measuring the total MP or MMP activity in the saliva sample; and
(c) comparing the total MP or MMP activity in the saliva sample to previously-determined baseline salivary MP or MMP activity in that individual;
wherein a significant increase in the individual's salivary MP or MMP activity relative to the baseline salivary MP or MMP activity provides an indication of increased risk.

27. The method of claim 26, wherein the baseline salivary MP or MMP activity is determined by measuring salivary MP or MMP activity in the individual between 6 and 18 weeks gestation, and the saliva sample of (a) is obtained after 18 weeks gestation.

28. The method of claim 26, wherein the baseline salivary MP or MMP activity is determined by measuring salivary MP or MMP activity in the individual between 12 and 18 weeks gestation, and the saliva sample of (a) is obtained after 18 weeks gestation.

29. The method of any one of claims 25-28, wherein a significant increase in the individual's salivary MP or MMP activity relative to the individual's baseline salivary MP or MMP activity is a statistically-significant increase.

30. The method of any one of claims 25-28, wherein a significant increase in the individual's salivary MP or MMP activity relative to the individual's baseline salivary MP or MMP activity is at least a two-fold increase.

31. The method of any one of claims 25-28, wherein a significant increase in the individual's salivary MP or MMP activity relative to the individual's baseline salivary MP or MMP activity is at least a three-fold increase.

32. A method for determining whether a pregnant individual is at increased risk of preterm delivery induced by premature rupture of the fetal membranes, comprising:
(a) obtaining a saliva sample from the individual;
(b) measuring the total proteinase activity in the saliva sample; and
(c) comparing the total proteinase activity in the saliva sample to a normal salivary proteinase activity;
wherein a significant increase in the individual's salivary proteinase activity relative to the normal salivary proteinase activity provides an indication of increased risk.

33. A method for determining whether a pregnant individual is at increased risk of preterm delivery induced by premature rupture of the fetal membranes, comprising:
(a) obtaining a saliva sample from the individual;
(b) measuring the total proteinase activity in the saliva sample; and
(c) comparing the total proteinase activity in the saliva sample to previously-determined baseline salivary proteinase activity in that individual;
wherein a significant increase in the individual's salivary proteinase activity relative to the baseline salivary proteinase activity provides an indication of increased risk.

34. The method of claim 33, wherein the baseline salivary proteinase activity is determined by measuring salivary proteinase activity in the individual between 6 and 18 weeks gestation, and the saliva sample of (a) is obtained after 18 weeks gestation.

35. The method of claim 33, wherein the baseline salivary proteinase activity is determined by measuring salivary proteinase activity in the individual between 12 and 18 weeks gestation, and the saliva sample of (a) is obtained after 18 weeks gestation.

36. The method of any one of claims 32-35, wherein a significant increase in the individual's salivary proteinase activity relative to the individual's baseline salivary proteinase activity is a statistically-significant increase.

37. The method of any one of claims 32-35, wherein a significant increase in the individual's salivary proteinase activity relative to the individual's baseline salivary proteinase activity is at least a two-fold increase.

38. The method of any one of claims 32-35, wherein a significant increase in the individual's salivary proteinase activity relative to the individual's baseline salivary proteinase activity is at least a three-fold increase.
